(19) United States
(12) Patent Application Publication
(10) Pub. No.: US 2024/0101701 A1
(43) Pub. Date: Mar. 28, 2024

(54) ANTIBODY-DERIVED T CELL ACTIVATING TECHNOLOGIES

(71) Applicant: Health Research, Inc., Buffalo, NY (US)

(72) Inventors: Leonid CHERKASSKY, Buffalo, NY (US); Richard KOYA, Williamsville, NY (US); Kunle ODUNSI, Williamsville, NY (US); Ben K. SEON, Williamsville, NY (US)

(21) Appl. No.: 18/546,394

(22) PCT Filed: Feb. 17, 2022
(86) PCT No.: PCT/US2022/016845

§ 371 Date: Aug. 14, 2023

Related U.S. Application Data
(60) Provisional application No. 63/150,723, filed on Feb. 18, 2021.

(51) Int. Cl. C07K 16/28; A61K 39/00; C07K 14/705; C07K 14/725; C12N 5/0783

(52) U.S. Cl. CPC C07K 16/2896; A61K 39/4611; A61K 39/4631; A61K 39/464429; C07K 14/7051; C07K 14/70521; C07K 14/70578; C12N 5/0636; A61K 2239/29; C07K 2317/622; C07K 2319/02; C07K 2319/03; C12N 2510/00

(57) ABSTRACT

Antibody derivatives are provided as binding partners. The binding partners bind to a one or a combination of antigens that include antigens present CD24, CD105 (endoglin), CD79 Beta (CD79b), and an antigen present in a CD3 T cell co-receptor. The antibody derivatives include single chain variable fragments (scFvs), Bi-specific T-cell engagers (BiTEs). Also provided are modified cells that express the binding partners, modified cells that secrete the binding partners, expression vectors that encode the binding partners, and methods of using the binding partners for treatment of a variety of cancers, autoimmune diseases, and modification of immune responses mounted to transplanted organs.

### CROSS REFERENCE TO RELATED APPLICATIONS

This application claims priority to U.S. provisional application No. 63/150,723, filed Feb. 18, 2021, the disclosure of which is incorporated herein by reference.

### BACKGROUND

There is an ongoing and unmet need for improved compositions and methods for targeting cancers, and other immune-based clinical applications such as limiting autoimmune disease and preventing rejection of organ transplants. The present disclosure is pertinent to this need.

### SEQUENCE LISTING

The instant application contains a Sequence Listing which has been submitted electronically in ASCII format and is hereby incorporated by reference in its entirety. Said ASCII copy, created on Feb. 15, 2022, is named 003551_01031.txt, and is 14,777 bytes in size.

### BRIEF DESCRIPTION OF THE FIGURES

FIG. 1. Graph representing cytotoxicity of chimeric antigen receptor (CAR) T cells targeted to CD24. The irrelevant antigen is CD19. The tumor cells are pancreatic cancer cells.

FIG. 2. Graphs representing enzyme-linked immunoassay (ELISA) sandwich assay measuring interleukin 2 (IL-2) (left panel) and interferon alpha (IFN-α) (right panel) secretion by CAR T cells targeting CD24. The irrelevant antigen is CD19. The tumor cells are pancreatic cancer cells.

FIG. 3. Graph representing cytoxocity of CAR T cells towards endoglin (CD105) expressing cancer cells. The cancer cells are Nalm-6 cells.

FIG. 4. Graphs representing ELISA sandwich assay measuring interleukin 2 (IL-2) (left panel) and interferon alpha (IFN-a) (right panel) secretion by CAR T cells targeting endoglin. The cancer cells are Nalm-6 cells.

### SUMMARY

The disclosure provides in various embodiments derivatives of antibodies, referred to herein as binding partners, compositions comprising the binding partners, cells that are modified to express and/or secrete certain binding partners, and polynucleotides encoding the binding partners. The binding partners and cells modified to express them or useful for treating cancer and other disorders as described herein. In embodiments, the described binding partners activate T cells, and have the ability to establish memory which allows for long-lasting functionality. Binding partner derivatives described herein are based in part on the following antibodies:
1: Anti-CD24 mAb SN3 (Clone A5-2H10) IgG1-kappa
2: Anti-CD24 mAb SN3a (SN3 Alpha) (Clone J3-3D3) IgG1-kappa
3: Anti-CD24 mAb SN3b (SN3 Beta) (Clone N6-3G5-2G1) IgM-kappa
4: Anti-CD105 mAb SN6h (Clone G4-2C2) IgG1-kappa
5: Anti-CD79b (CD79 Beta) mAb SN8 (3A2-2E7-1F5) IgG1-kappa.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The present disclosure relates to antibody-derived constructs, as follows. CARs are synthetic receptors that retarget T cells to tumor surface antigens. This is a two-part synthetic receptor typically composed of a) an antibody-derived single chain variable fragments (scFvs), which is responsible for antigen binding by the CAR, and b) an intracellular portion of the receptor encoding T cell signaling domains, which activate T cells upon antigen binding. T cell activation domains typically encode for both CD3ζ fused in tandem to costimulatory signaling such as CD28 and/or 4-1BB to achieve optimal proliferation and T cell survival upon activation. CAR constructs are typically synthesized and cloned into a retroviral or lentiviral plasmid backbone that can then be used for viral production and T cell transduction to generate CAR T cells for therapeutic applications. BiTEs similarly incorporate antibody-derived scFvs and are composed of a tumor-targeted scFv providing tumor antigen target specificity, linked in tandem to a T cell specific scFv, which provides T cell activation (typically an anti-CD3 scFv). Ribosomal skip sites are incorporated at appropriate locations. Since CARs and BiTEs utilize cancer antigen binding domains derived from antibodies, an efficient strategy to generate novel CARs is converting existing therapeutic antibodies. The described constructs may contain additional amino acids, such as any suitable amino acid sequence used for protein purification.

The described antibody-derived T cell activating technologies represent a critical and unmet need. In certain embodiments, they target cancer antigens ideally suited for CAR T cell development for the following reasons: 1) expression that is of high intensity and homogeneity to maximize tumor cell targeting, 2) relative overexpression on cancer tissues relative to normal tissues or else expressed on noncritical normal tissue, which is essential to limit ‘on-target off-tumor’ toxicity, and 3) targets are drivers of cancer development and aggressiveness, therefore surviving antigen-negative escape variants are less likely to grow out and mediate recurrence. The targets these antibodies are specific for are as follows:

Endoglin (ENG, CD105) is essential for tumor vessel angiogenesis and is selectively overexpressed on vascular and lymphatic endothelium in pancreatic, HCC, ovarian, breast and colorectal tumors with limited expression on normal endothelium.

CD24 (heat stable antigen or small-cell lung carcinoma cluster 4 antigen) is overexpressed in many solid tumors and promotes tumor invasiveness. Its expression on cancer stem cells and its function in triple-negative breast cancer and ovarian cancer as an innate immune checkpoint signal is responsible for immune evasion of macrophage-mediated phagocytosis has highlighted it as a particularly attractive target.

CD79b (B-cell receptor-associated protein) is a critical receptor for successful B cell development that remains highly expressed on most non-Hodgkin lymphomas (NHL) while normal tissue expression is limited to mature B cells—a dispensable tissue. Furthermore, this antigen is retained on B cell lymphomas independent of loss of CD19 highlighting its usefulness in treating disease relapse due to CD19 antigen loss or as an upfront strategy in combination with CD19 CAR T cell therapy to prevent antigen immune escape. This invention includes CAR T cells and BiTEs derived from a CD79b targeted antibody. This disclosure includes CAR T cells, single chain variable fragments (scFvs), BiTEs, as well as CAR T cells secreting scFvs and/or BiTEs for clinical application to the treatment of cancers, autoimmune diseases, and modifying immune response to transplanted organs.

As discussed above, the disclosure related in part to derivatives of anti-CD24 mAb SN3, Anti-CD24 mAb SN3a, Anti-CD24 mAb SN3b, Anti-CD105 mAb SN6h, and Anti-CD79b mAb SNB. Thus, in certain embodiments, the disclosure provides a binding partner comprising heavy and light chain pairs. Binding partners may include a combination of heavy and light chain pairs, wherein optionally each the pairs bind with specificity to a different antigen. The antigens are thus selected from:
i) an antigen present in CD24;
ii) an antigen present in CD105 (endoglin);
iii) an antigen present in CD79 Beta (CD79b); or
iv) an antigen present in a CD3 T cell co-receptor.

In embodiments, the pair that binds with specificity to the antigen that is present in CD24 are (complementary determining regions (CDRs) for each heavy and light chain are shown in bold):
a) a heavy chain comprising the sequence:

(SEQ ID NO: 1)
QVQLKESGPGLVAPSQSLSITCTVSGFSLTSYGVHWV
RQPPGKGLEWLGVIWAGGSTNYNSALMSRLSISKDNS
KSQVFLKMNSLQTDDTAMYYCAVYYGYLFFAYWGQGT
LVTVSA;
and

b) a light chain comprising the sequence:

(SEQ ID NO: 2)
ENVLTQSPAIMSASPGEKVTMTCSASSSVSYMHWYQQK
STTSPKLWIYDTSKLASGVPGRFSGSGSGNSYSLTISS
MEAEDVATYYCFQGSGYPLTFGGGTKLEIK.

In an embodiment, the pair that binds with specificity to the antigen that is present in CD105 are:
c) a heavy chain comprising the sequence:

(SEQ ID NO: 3)
EVMLVESGGGLVKPGGSLKLSCEASGFTFSSYAMSWVR
QTPEKKLEWVAIINSGGTYVYYTDSMKGRFTISRDNAK
NTLYLQMSSLRSEDTAMYYCARRNYDGSYGFYFDYWGQ
GTTLTVSS;
and

d) a light chain comprising the sequence:

(SEQ ID NO: 4)
DIVMTQSPSSLSVSAGEKVTMNCKSSQSLLNSGNQKNY
LAWHQQKPGQPPKLLIYGASTRESGVPDRFTGSGSGTD
FTLTISSVQAEDLAVYYCQNDHSYPYTFGGGTKLEIK.

In an embodiment, the pair binds with specificity to the antigen that is present in CD79b are:
e) a heavy chain comprising the sequence:

(SEQ ID NO: 5)
QVQLQQSGAELMKPGASVKISCKATGYTFSSYWIEWVKQRPGH
GLEWIGEILPGGGDTNYNEIFKGKATFTADTSSNTAYMQLSSL
TSEDSAVYYCTRRVPVYFDYWGQGTTLTVSS;
and

f) a light chain comprising the sequence:

(SEQ ID NO: 6)
DIVLTQSPASLAVSLGQRATISCKASQSVDYDGDSFLNWYQQK
PGQPPKLFIYAASNLESGIPARFSGSGSGTDFTLNIHPVEEED
AATYYCQQSNEDPLTFGAGTELELK;
or

In an embodiment, the pair that binds to the antigen that is present in CD3 are
g) a heavy chain sequence comprising the sequence:

(SEQ ID NO: 7)
EVQLQQSGPELVKPGASMKISCKASGYSFTGYTMNWVKQSHGK
NLEWMGLINPYKGVSTYNQKFKDKATLTVDKSSSTAYMELLSL
TSEDSAVYYCARSGYYGDSDWYFDVWGQGTTLTVFS;
and

h) a light chain comprising the sequence:

(SEQ ID NO: 8)
MDIQMTQTTSSLSASLGDRVTISCRASQDIRNYLNWYQQKPD
GTVKLLIYYTSRLHSGVPSKFSGSGSGTDYSLTISNLEQEDI
ATYFCQQGNTLPWTFAGGTKLEIK.

In one embodiment, the disclosure provides:
i) a single-chain variable fragment (scFv), and wherein the heavy and light chain pair binds with specificity to an antigen present in the CD24, the CD105, or the CD79b.

In another embodiment, the disclosure provides:
ii) a Bi-specific T-cell engager (BiTE), wherein the BiTE comprises one pair of heavy and light chains that bind to an antigen in the CD24, the CD105, or the CD79b; and one pair of heavy and light chains that bind to an antigen present in the CD3.

In an embodiment, an ScFv is a component of a chimeric antigen receptor (CAR), the scFv being present in a contiguous polypeptide that further comprises:
a CD3zeta chain;
and at least one of:
a 4-1BB costimulatory domain, said domain optionally further comprising a CD8 co-receptor hinge sequence; or
a CD28 costimulatory signaling sequence.

The disclosure includes polynucleotides that encode the described binding partners, and expression vectors as further described below. The disclosure also includes modified eukaryotic cells that contain the polynucleotides, and express the described binding partners. The eukaryotic cells include but are not necessarily limited to any type of T Cell. In embodiments, the modified T cell expresses an scFv, which may or may not secrete the ScFv. Modified T cells that express chimeric antigen receptors are also included, as discuss further below. In a non-limiting embodiment, the disclosure provides a modified T cell that expresses a CAR, and also expresses an scFv that is not part of the CAR. That ScFv that is not part of the CAR may or may not be secreted by the T cell.

In another approach, the disclosure provides a method comprising obtaining T cells from an individual and modifying the T cells so that the T cells express a binding partner that is an scFv, a CAR, or a combination thereof. The modified T cells may be used in autologous or allogenic therapies.

Notwithstanding the foregoing description, binding partners of this disclosure can be provided as intact immunoglobulins, or as fragments of immunoglobulins, including but not necessarily limited to antigen-binding (Fab) fragments, Fab′ fragments, (Fab′)₂ fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), isolated CDR regions, the aforementioned scFvs, and other antibody fragments that retain antigen binding function. As described above, in embodiments, one or more binding partners are provided as a component of a BiTE or a CAR. In another embodiment, the binding partners can be provided as bispecific killer cell engager (BiKE). Thus, the binding partners are in certain examples multivalent. In embodiments, a tri-specific binding partner is provided. In embodiments, leukocytes, including but not necessarily T cells, express at least a segment of one or more binding partners in the form of a CAR. In embodiments, a multi-valent binding partner includes one binding component, such as a paratope, that confers specificity to a particular target on a desired cell type, such as any cancer cell marker.

In certain embodiments, a binding partner of this disclosure, such as an ScFv, can be provided with additional, contiguous amino acids. The additional amino acids may impart a function to the binding partner, such as surface display or secretion.

For CARs, the binding partners include additional components, such as a costimulatory signaling sequence, an optional hinge region, and a CD3zeta chain. Any suitable costimulatory sequences may be used, non-limiting examples of which include the 4-1BB costimulatory signal sequence, and the CD28 costimulatory sequence. In a non-limiting embodiment, a construct that comprises a 4-1BB costimulatory signaling preceded by a CD8 coreceptor hinge region and followed by CD3zeta chain comprises or consists of the sequence:

(SEQ ID NO: 9)
AAAPTTTPAPRPPTPAPTIASQPLSLRPEACRPAAGGAVHTR
GLDFACDIYIWAPLAGTCGVLLLSLVITLYCNKRGRKKLLYI
FKQPFMRPVQTTQEEDGCSCRFPEEEEGGCELRVKFSRSAEP
PAYQQGQNQLYNELNLGRREEYDVLDKRRGRDPEMGGKPRRK
NPQEGLYNELQKDKMAEAYSEIGMKGERRRGKGHDGLYQGLS
TATKDTYDALHMQALPPR.

In another embodiment, a CD28 costimulatory signaling followed by CD3zeta chain comprises or consists of the sequence

(SEQ ID NO: 10)
AAAIEVMYPPPYLDNEKSNGTIIHVKGKHLCPSPLFPGPSKP
FWVLVVVGGVLACYSLLVTVAFIIFWVRSKRSRLLHSDYMNM
TPRRPGPTRKHYQPYAPPRDFAAYRSRVKFSRSAEPPAYQQG
QNQLYNELNLGRREEYDVLDKRRGRDPEMGGKPRRKNPQEGL
YNELQKDKMAEAYSEIGMKGERRRGKGHDGLYQGLSTATKDT
YDALHMX.

For BiTEs, one binding partner binds comprises a segment that binds with specificity to a selected cancer antigen (e.g., CD24, Endoglin, or CD79b) linked to another 15 segment targeting a CD3 T cell co-receptor. This BiTE may be delivered systemically or locally delivered as a secreted protein by a T cell that does or does not also express a CAR.

In embodiments, binding partners of this disclosure may comprise linking sequences. As a non-limiting example, an ScFv may comprise a linker that links segments comprising paratopes to one another. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, and 20 amino acids. In an example, the linker may be the glycine-serine-alanine linker G₄SA₃ or a glycine-serine linker (G₄S)₄ linker.

In embodiments, such as for proteins that are produced as a fusion protein, a peptide linker may be used, and may comprise a cleavable or non-cleavable linker. In embodiments, the peptide linker comprises any self-cleaving signal. In embodiments, the self-cleaving signal may be present in the same open reading frame (ORF) as the ORF that encodes the binding partner. A self-cleaving amino acid sequence is typically about 18-22 amino acids long. Any suitable sequence can be used, non-limiting examples of which include:

T2A
(SEQ ID NO: 11)
(EGRGSLLTCGDVEENPGP;
P2A
(SEQ ID NO: 12)
(ATNFSLKQAGDVENPGP;
E2A
(SEQ ID NO: 13)
(QCTNYALKLAGDVESNPGP
and
F2A
(SEQ ID NO: 14)
(VKQTLNFDLKLAGDVESNPGP.

In embodiments, a binding partner may include a cellular localization signal, or a secretion signal. In embodiments, binding partner may comprises a transmembrane domain, and thus may be trafficked to, and anchored in a cell membrane. For secretion, any suitable secretion signal can be used and many are known in the art. In non-limiting embodiments, the secretion signal comprises MALPVTALLLPLALLLHA (SEQ ID NO:15) METDTLLLWVLLLWVPGSTG (SEQ ID NO:16) or MGWSCIILFLVATATGVHSD (SEQ ID NO:17). For example, a signal peptide may be used to allow surface expression of an ScFv. In another embodiment, a binding partner is designed so that it may be secreted by a modified cell, including but not limited to a T cell.

In embodiments, binding partners of this disclosure may comprise or may not comprise a constant region, e.g., an Fc region. Any isotype of constant region can be included. Binding partners that comprise a constant region may be particularly adapted for antibody-dependent cell mediated cytotoxicity (ADCC) and thus may function to kill targeted cells by complement-mediated responses and/or by cell-mediated responses by any of a variety of effector cells.

In embodiments, binding partners described herein are used to carry drugs or toxins, and thus the binding partners may be provided as immunotoxins, or in the form of antibody-drug conjugates (ADCs).

Any binding partner described herein may be fully or partially humanized. Techniques for humanization of antibodies are known in the art and can be adapted for use in the present disclosure. In embodiments, humanization may be performed, for example, by CDR-grafting. In embodiments, for humanization or to otherwise improve a characteristic of the binding partners, one or more amino acids in a variable region can be changed. In embodiments, one or more amino acids in a framework region can be changed.

For therapeutic approaches, in certain embodiments, binding partners may be delivered as mRNA or DNA polynucleotides that encode the binding partners. It is considered that administering a DNA or RNA encoding any binding partner described herein is also a method of delivering such binding partners to an individual or one or more cells, provided the DNA is transcribed, and/or the RNA is translated. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retroviral vector. In embodiments, the retroviral vector is adapted from a murine Moloney leukemia virus (MLV) or a lentiviral vector may be used, such as a lentiviral vector adapted from human immunodeficiency virus type 1 (HIV-1).

In an embodiment, an oncolytic viral vector is used. Oncolytic viruses (OVs), including vaccinia (OVV), mediate anticancer effects by both direct oncolysis and stimulation of innate immune responses through production of damage-associated molecular patterns (DA1VIPs) and the presence of virus-derived pathogen-associated molecular patterns (PAMPs), leading to increased type I interferon production. Additionally, OVV-mediated oncolysis may facilitate the direct acquisition of tumor-derived antigens by host antigen-presenting cells within the tumor microenvironment, thereby leading to improved T cell priming as well as coordination of the effector phase of antitumor immune responses. In alternative embodiments, a recombinant adeno-associated virus (AAV) vector may be used. In certain embodiments, the expression vector is a self-complementary adeno-associated virus (scAAV).

In embodiments, gene editing approach may be used to modify cells to express a described binding partner. In embodiments, a guide-directed nuclease may be used, non-limiting examples of which include CRISPR Type I, II and III systems. In embodiments, transposon based system may be used, a non-limiting example of which comprises modified piggyBac (PB) and Sleeping Beauty (SB) DNA transposons.

Pharmaceutical formulations containing binding partners are included in the disclosure, and can be prepared by mixing them with one or more pharmaceutically acceptable carriers. Pharmaceutically acceptable carriers include solvents, dispersion media, isotonic agents and the like. The carrier can be liquid, semi-solid, e.g. pastes, or solid carriers. Examples of carriers include water, saline solutions or other buffers (such as phosphate, citrate buffers), oil, alcohol, proteins (such as serum albumin, gelatin), carbohydrates (such as monosaccharides, disaccharides, and other carbohydrates including glucose, sucrose, trehalose, mannose, mannitol, sorbitol or dextrins), gel, lipids, liposomes, resins, porous matrices, binders, fillers, coatings, stabilizers, preservatives, liposomes, antioxidants, chelating agents such as EDTA; salt forming counter-ions such as sodium; non-ionic surfactants such as TWEEN, PLURONICS or polyethylene glycol (PEG), or combinations thereof.

In embodiments, an effective amount of one or more binding partners is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight and gender of the patient, desired duration of treatment, method of administration, time and frequency of administration, drug combination(s), reaction sensitivities, and/or tolerance/response to therapy.

Binding partners and pharmaceutical compositions comprising the binding partners can be administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. Intra-tumor injections may also be used. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be therapeutically indicated.

In embodiments, the individual in need of a composition of this disclosure has been diagnosed with or is suspected of having cancer. In embodiments, the cancer is a solid or liquid tumor. In embodiments, the cancer is renal cell carcinoma, breast cancer, prostate cancer, pancreatic cancer, lung cancer, liver cancer, ovarian cancer, cervical cancer, colon cancer, esophageal cancer, glioma, glioblastoma, or another brain cancer, stomach cancer, bladder cancer, testicular cancer, head and neck cancer, melanoma or another skin cancer, any sarcoma, including but not limited to fibrosarcoma, angiosarcoma, adenocarcinoma, and rhabdomyosarcoma, and any blood cancer, including all types of leukemia, lymphoma, and myeloma.

In embodiments, administering one or more binding partners, including but not necessarily in a pharmaceutical formulation, to an individual in need thereof, exhibits an improved activity relative to a control. In an embodiment, the control comprises different antibodies, a different form of the same antibodies/binding partner, or antibodies/binding partners that are delivered without adding additional agents.

A composition of this disclosure, such as a pharmaceutical formulation, can contain only one, or more than one binding partner, and thus combinations of different binding partners are included. Likewise, one or more binding partners can be combined with any other therapeutic agent, non-limiting examples of which include conventional chemotherapeutic agents, and immune checkpoint inhibitors, the latter of which are known in the art, and target CTLA4, PD-1, or PD-L1. Thus, the disclosure includes combination therapy using one or more described binding partners and any of CTLA-4 inhibitors, PD-1 inhibitors and PD-L1 inhibitors. As non-limiting examples, anti-PD-1 agents include Pembrolizumab and Nivolumab. Anti-PD-L1 examples include Avelumab and Atezolizumab. An anti-CTLA-4 example is Ipilimumab. The binding partners may also be combined with any form of adoptive immunotherapy.

The following Examples are intended to illustrate but not limit the disclosure.

### EXAMPLES

The following examples use a murine stem cell virus retroviral vector encoding the described CAR T cells. The sequences of the CARs is as described in the detailed description. Each CAR contained an ScFv containing the described heavy and light chain sequences targeted to the described antigen as in each figure, the described 4-1BB costimulatory domain sequence with a transmembrane domain, the described CD28 derived hinge sequence with a transmembrane domain, and the described CD3zeta chain sequence.

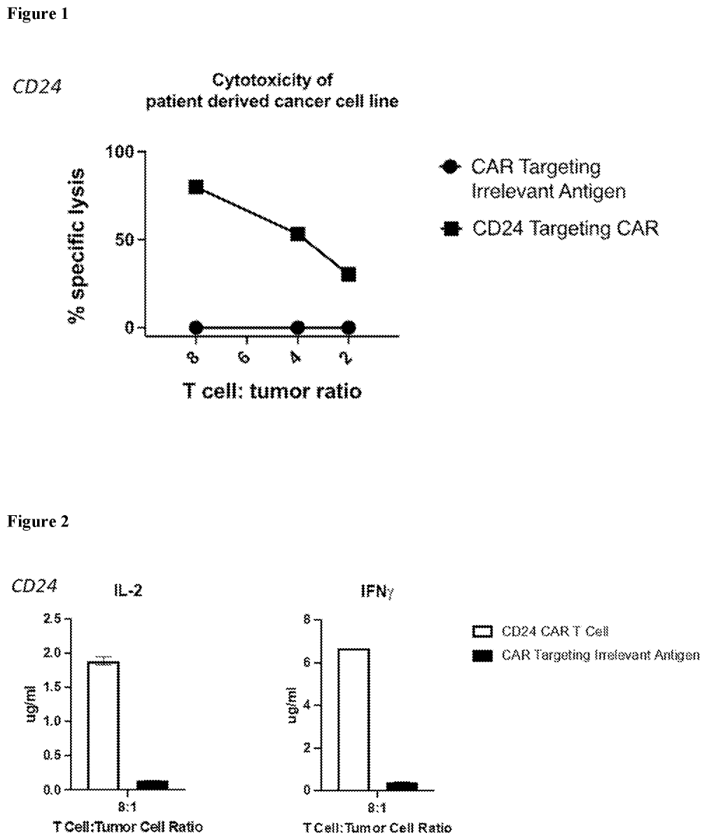

FIG. 1 provides a graph representing cytotoxicity of CAR T cells targeted to CD24. The irrelevant antigen is CD19. The data demonstrate that CAR T cells targeting CD24 lyse CD24 expressing cancer cells. The data shown in the graph were obtained using a flow cytometry-based cytolytic assay using CAR T cells specific for CD24 or the irrelevant antigen in coculture with CD24 expressing patient-derived cancer cell line. The tumor cells are pancreatic cancer cells.

FIG. 2 provides graphs showing data from ELISA assays for cytokine responses from the CAR T cells targeted to CD24 and the irrelevant antigen control as indicated. The data show that CAR T cells targeting CD24 secrete Th1 cytokines IL-2 and IFNγ. The data were obtained using the ELISA assay on supernatants of cocultures containing CAR T cells (specific for CD24 antigen or control irrelevant antigen) and CD24 expressing patient-derived cancer cell line that are pancreatic cancer cells.

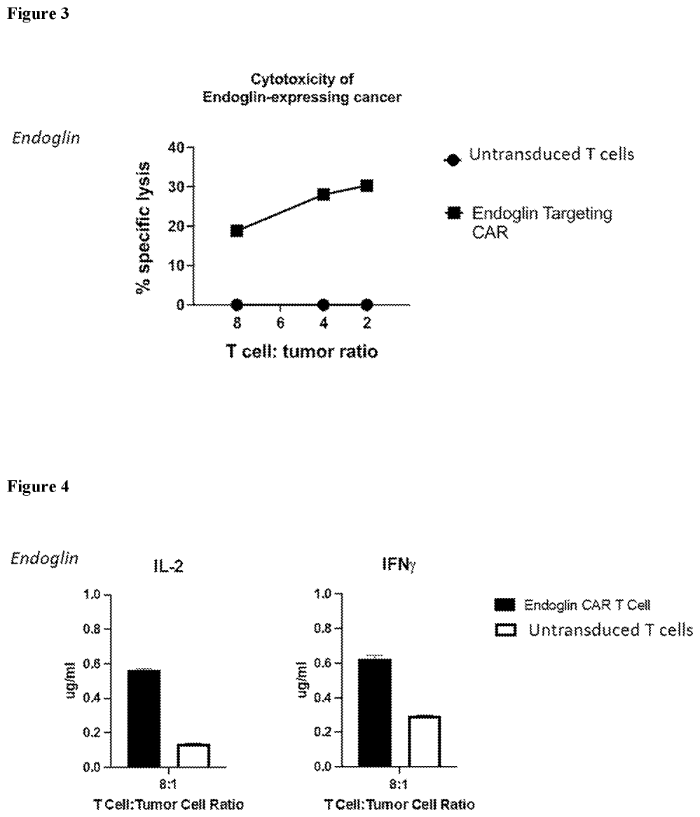

FIG. 3 provides a graph representing cytoxocity of CAR T cells towards endoglin (CD105) expressing cancer cells with non-transduced T cells as a control. The cancer cells are Nalm-6 cells. The data show that CAR T cells targeting endoglin lyse endoglin expressing cancer cells. The data were obtained using a flow cytometry-based cytolytic assay following CAR T cell (specific for the endoglin antigen) in co-culture with the endoglin expressing cancer cell line.

FIG. 4 provides graphs representing ELISA sandwich assay measuring interleukin 2 (IL-2) (left panel) and interferon alpha (IFN-a) (right panel) secretion by CAR T cells targeting endoglin. The cancer cells are Nalm-6 cells. The data show that CAR T cells targeting endoglin secrete Th1 cytokines IL-2 and IFNγ. The data were obtained using supernatants of cocultures containing CAR T cells (specific for endoglin antigen or control as indicated) and an endoglin expressing patient-derived cancer cell line.

Although the subject matter of this disclosure has been described above in terms of certain embodiments/examples, other embodiments/examples, including embodiments/examples that do not provide all of the benefits and features set forth herein, are also within the scope of this disclosure.

1. A binding partner comprising heavy and light chain pairs, wherein optionally each of said pairs bind with specificity to a different antigen, wherein the antigens are selected from:
i) an antigen present in CD24;
ii) an antigen present in CD105 (endoglin);
iii) an antigen present in CD79 Beta (CD79b); or
iv) an antigen present in a CD3 T cell co-receptor.

2. The binding partner of claim 1:
wherein the pairs that bind with specificity to the antigen that is present in CD24 are:
a) a heavy chain comprising the sequence:
(SEQ ID NO: 1)
QVQLKESGPGLVAPSQSLSITCTVSGFSLTSYGVHWVRQP
PGKGLEWLGVIWAGGSTNYNSALMSRLSISKDNSKSQVFL
KMNSLQTDDTAMYYCAVYYGYLFFAYWGQGTLVTVSA;
and
b) a light chain comprising the sequence:
(SEQ ID NO: 2)
ENVLTQSPAIMSASPGEKVTMTCSASSSVSYMHWYQQKSTTSPKL
WIYDTSKLASGVPGRFSGSGSGNSYSLTISSMEAEDVATYYCFQG
SGYPLTFGGGTKLEIK;
or
wherein the pairs that bind with specificity to the antigen that is present in CD105 are:
c) a heavy chain comprising the sequence:
(SEQ ID NO: 3)
EVMLVESGGGLVKPGGSLKLSCEASGFTFSSYAMSWVRQTPEKKLE
WVAIINSGGTYVYYTDSMKGRFTISRDNAKNTLYLQMSSLRSEDTA
MYYCARRNYDGSYGFYFDYWGQGTTLTVSS;
and
d) a light chain comprising the sequence:
(SEQ ID NO: 4)
DIVMTQSPSSLSVSAGEKVTMNCKSSQSLLNSGNQKNYLAWHQQKP
GQPPKLLIYGASTRESGVPDRFTGSGSGTDFTLTISSVQAEDLAVY
YCQNDHSYPYTFGGGTKLEIK;
or
wherein the pairs that bind with specificity to the antigen that is present in CD79b are:
e) a heavy chain comprising the sequence:
(SEQ ID NO: 5)
QVQLQQSGAELMKPGASVKISCKATGYTFSSYWIEWVKQRPGH
GLEWIGEILPGGGDTNYNEIFKGKATFTADTSSNTAYMQLSSL
TSEDSAVYYCTRRVPVYFDYWGQGTTLTVSS;
and
f) a light chain comprising the sequence:
(SEQ ID NO: 6)
DIVLTQSPASLAVSLGQRATISCKASQSVDYDGDSFLNWYQQKPGQPP
KLFIYAASNLESGIPARFSGSGSGTDFTLNIHPVEEEDAATYYCQQSN
EDPLTFGAGTELELK;
or
wherein the pairs that bind with specificity to the antigen that is present in CD3 are
g) a heavy chain sequence comprising the sequence:
(SEQ ID NO: 7)
EVQLQQSGPELVKPGASMKISCKASGYSFTGYTMNWVKQSHGKNL
EWMGLINPYKGVSTYNQKFKDKATLTVDKSSSTAYMELLSLTSED
SAVYYCARSGYYGDSDWYFDVWGQGTTLTVFS;
and
h) a light chain comprising the sequence:
(SEQ ID NO: 8)
MDIQMTQTTSSLSASLGDRVTISCRASQDIRNYLNWYQQKPDGTVK
LLIYYTSRLHSGVPSKFSGSGSGTDYSLTISNLEQEDIATYFCQQG
NTLPWTFAGGTKLEIK.

3. The binding partner of claim 1, wherein the binding partner comprises:
i) a single-chain variable fragment (scFv), and wherein the heavy and light chain pair binds with specificity to an antigen present in the CD24, the CD105, or the CD79b; or
ii) a Bi-specific T-cell engager (BiTE), and wherein the BiTE comprises one pair of heavy and light chains that bind to an antigen in the CD24, the CD105, or the CD79b; and
one pair of heavy and light chains that bind to an antigen present in the CD3.

4. The binding partner of claim 3, wherein the binding partner comprises the scFv.

5. The binding partner of claim 4, wherein the scFv is a component of a chimeric antigen receptor (CAR), the scFv being present in a contiguous polypeptide that further comprises:
a CD3zeta chain;
and at least one of:
a 4-1BB costimulatory domain, said domain optionally further comprising a CD8 co-receptor hinge sequence; or
a CD28 costimulatory signaling sequence.

6. The binding partner of claim 3, wherein the binding partner comprises the BiTE.

7. The binding partner of claim 3, wherein the scFv comprises a cell surface display signal, or a secretion signal.

8. A polynucleotide encoding a binding partner of claim 1.

9. The polynucleotide of claim 8, wherein the polynucleotide is a component of an expression vector, said expression vector optionally comprising a retroviral vector.

10. A modified eukaryotic cell comprising the polynucleotide of claim 9.

11. The modified eukaryotic cell of claim 10, wherein the eukaryotic cell is a T Cell.

12. The modified eukaryotic cell of claim 11, wherein the T cell expresses the scFv, and wherein the ScFv is optionally secreted by the T cell.

13. The modified eukaryotic cell of claim 11, wherein the T cell expresses the CAR.

14. The modified eukaryotic cell of claim 13, wherein T cell that expresses the CAR further expresses an scFv that is not part of the CAR, and wherein the scFv that is not part of the CAR is optionally secreted by the T cell.

15. A method comprising obtaining T cells from an individual and modifying the T cells so that the T cells express a binding partner that is an scFv, a CAR, or a combination thereof, according to claim 1.

16. The method of claim 15, further comprising administering modified T cells to an individual in need thereof, and wherein optionally the T cells were isolated from said individual prior to modification to express the scFv, the CAR, or the combination thereof.

17. The method of claim 16, wherein the T cells express the scFv, and wherein the scFv is secreted by the T cells.

18. A method comprising introducing into an individual in need thereof a composition comprising an scFv, or a BiTE, as in claim 3.

19. The method of claim 18, wherein the method comprises administering the composition comprising the scFv.

20. The method of claim 18, wherein the method comprises administering the BiTE.

21. A pharmaceutical composition comprising an scFv or a BiTE according to claim 3.


SEQUENCE LISTING (17 sequences)

SEQ ID NO: 1 - protein, 117 aa, Mus musculus
QVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCAVYYGYLFFAYWGQGTLVTVSA

SEQ ID NO: 2 - protein, 106 aa, Mus musculus
ENVLTQSPAIMSASPGEKVTMTCSASSSVSYMHWYQQKSTTSPKLWIYDTSKLASGVPGRFSGSGSGNSYSLTISSMEAEDVATYYCFQGSGYPLTFGGGTKLEIK

SEQ ID NO: 3 - protein, 122 aa, Mus musculus
EVMLVESGGGLVKPGGSLKLSCEASGFTFSSYAMSWVRQTPEKKLEWVAIINSGGTYVYYTDSMKGRFTISRDNAKNTLYLQMSSLRSEDTAMYYCARRNYDGSYGFYFDYWGQGTTLTVSS

SEQ ID NO: 4 - protein, 113 aa, Mus musculus
DIVMTQSPSSLSVSAGEKVTMNCKSSQSLLNSGNQKNYLAWHQQKPGQPPKLLIYGASTRESGVPDRFTGSGSGTDFTLTISSVQAEDLAVYYCQNDHSYPYTFGGGTKLEIK

SEQ ID NO: 5 - protein, 117 aa, Mus musculus
QVQLQQSGAELMKPGASVKISCKATGYTFSSYWIEWVKQRPGHGLEWIGEILPGGGDTNYNEIFKGKATFTADTSSNTAYMQLSSLTSEDSAVYYCTRRVPVYFDYWGQGTTLTVSS

SEQ ID NO: 6 - protein, 111 aa, Mus musculus
DIVLTQSPASLAVSLGQRATISCKASQSVDYDGDSFLNWYQQKPGQPPKLFIYAASNLESGIPARFSGSGSGTDFTLNIHPVEEEDAATYYCQQSNEDPLTFGAGTELELK

SEQ ID NO: 7 - protein, 122 aa, Mus musculus
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 8 - protein, 108 aa, Mus musculus
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 9 - protein, 228 aa, artificial sequence
  synthetic CD3zeta chain sequence
AAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 217 aa, artificial sequence
  synthetic protein
  misc_feature (217)..(217): Xaa can be any naturally occurring amino acid
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMX

SEQ ID NO: 11 - protein, 18 aa, artificial sequence
  synthetic skipping sequence
EGRGSLLTCGDVEENPGP

SEQ ID NO: 12 - protein, 17 aa, artificial sequence
  synthetic skipping sequence
ATNFSLKQAGDVENPGP

SEQ ID NO: 13 - protein, 19 aa, artificial sequence
  synthetic skipping sequence
QCTNYALKLAGDVESNPGP

SEQ ID NO: 14 - protein, 20 aa, artificial sequence
  synthetic skipping sequence
VKQTLNFDLKLAGDVESNPG

SEQ ID NO: 15 - protein, 18 aa, artificial sequence
  synthetic secretion sequence
MALPVTALLLPLALLLHA

SEQ ID NO: 16 - protein, 20 aa, artificial sequence
  synthetic secretion sequence
METDTLLLWVLLLWVPGSTG

SEQ ID NO: 17 - protein, 20 aa, artificial sequence
  synthetic secretion sequence
MGWSCIILFLVATATGVHSD